(19) United States
(12) Patent Application Publication
(10) Pub. No.: US 2017/0181686 A1
(43) Pub. Date: Jun. 29, 2017

(54) BREATH TEST FOR ASSESSING LIVER DISEASE

(71) Applicant: EXALENZ BIOSCIENCE LTD., Modi'in (IL)

(72) Inventors: Gil GUGGENHEIM, Jerusalem (IL); Avraham HERSHKOWITZ, Nof Ayalon (IL); Yaron ILAN, Kefar Tavor (IL); Dan PERES, Tel Aviv (IL); Ilan BEN-OREN, Modi'in (IL)

(21) Appl. No.: 15/129,976

(22) PCT Filed: Apr. 1, 2015
(86) PCT No.: PCT/IL2015/050355

§ 371 Date: Sep. 28, 2016

Related U.S. Application Data
(60) Provisional application No. 61/973,856, filed on Apr. 2, 2014.

(51) Int. Cl. A61B 5/00; A61B 5/145; G01N 33/497; A61B 5/08

(52) U.S. Cl. CPC A61B 5/4244; A61B 5/082; A61B 5/7282; A61B 5/14532; A61B 5/14546; G01N 33/497

(57) ABSTRACT

There is provided herein a method and a device for measuring, using one or more breath sensors, a metabolic product of a fatty acid, a salt or a derivative thereof, in the subject's breath after administering to the subject an isotope labeled fatty acid, a salt or a derivative thereof, obtaining the subject's level of insulin, glucose, glucagon or a combination thereof and using a processing circuitry, evaluating the liver condition based on the subject's metabolic product of the fatty acid, salt or derivative thereof and the level of insulin, glucose, glucagon or a combination thereof.

### FIELD OF INVENTION

The present invention relates to the diagnosis of liver conditions.

### BACKGROUND OF THE INVENTION

Liver diseases can be caused by a variety of etiologies such as viral infection, metabolic diseases associated with obesity and metabolic syndrome, alcohol abuse and autoimmune disorders. The liver disease can be acute or develop into chronic conditions. The conditions can vary from mild disease to life threatening, and/or from mild through significant fibrosis and inflammation, ending in cirrhosis. Chronic liver disease and cirrhosis are currently the 12th leading cause of death, accounting for approximately 27,000 deaths annually (in the United States), with increasing numbers due to the onset of HCV (Hepatitis C Virus), obesity and metabolic syndrome epidemic. Moreover, alcoholic liver disease (ALD) is the most common cause of cirrhosis in the western world.

The condition of alcoholic fatty liver disease (AFLD) is defined as the presence of steatosis in addition to patient's alcohol consumption of over 20-30 g/day. The scope of AFLD ranges from alcoholic fatty liver (AFL) in early stages, to alcoholic steatohepatitis (ASH) in the advanced stages of AFLD. The complementary non-alcoholic fatty liver disease (NAFLD) is defined by the presence of predominantly macrovesicular hepatic steatosis or steatohepatitis in individuals who either do not consume any alcohol or consume alcohol in quantities that are not generally considered to be harmful to the liver. The histologic spectrum of NAFLD includes: Isolated non-alcoholic hepatic steatosis (NAFL) and non-alcoholic steatohepatitis (NASH). Alcoholic, as well as non-alcoholic fatty liver, AFL and NAFL respectively, are characterized by a fatty liver condition with no other histological abnormalities. Contrarily, the more severe conditions of alcoholic and non-alcoholic steatohepatitis (ASH and NASH) are characterized by steatosis along with other histologic findings, such as cytologic ballooning, Mallory's hyaline, inflammation and pericellular fibrosis. NASH and ASH have a similar pathogenesis and histopathology but a different etiology and epidemiology.

The minimal histologic criteria for diagnosing NASH/ASH are the presence of steatosis, inflammation and cytologic ballooning. Given the variable presence of these individual parameters, the presence of steatohepatitis is often made as an overall gestalt of the histological findings. The NASH activity score (NAS) was developed by the NIH NASH Clinical Research Network and ranges from 0 to 8. It is assembled from individual scores for steatosis, inflammation and cytologic ballooning. For example, the lobular inflammation in the NAS score is defined as follows (foci per 20× field): score 0 for no foci, score 1 for <2 foci, score 2 for 2-4 foci and score 3 for >4 foci per 20× field.

Recently a study was published concluding that lobular inflammatory scores had no association with those of portal chronic inflammation. From a mechanistic point, this observation implies distinct immunopathogenic processes in the lobules and portal tracts. This same separation can be inferred from the semiquantitative scoring systems developed for chronic hepatitis. In other words, the lobular inflammation has shown to have an important role in chronic liver disease in general.

The diagnosis of NAFLD takes place in the following steps: diagnosis of NASH (or, alternatively, fatty liver condition not diagnosed as NASH) should be made first. Then NAS is used to grade severity. In a reference study, NAS scores of 0-2 occurred in cases largely considered not diagnostic of NASH, scores of 3-4 were evenly divided among those considered not diagnostic, borderline, or positive for NASH, and scores of 5-8 occurred in cases that were largely considered diagnostic of NASH. Furthermore, histo-pathological based scores as well as other scores are being developed and may be used to assess and stage disease severity (e.g. a combination of the NAS and Fibrosis score).

NAFLD is the hepatic manifestation of the metabolic syndrome. The major risk factors associated with NAFLD are obesity, diabetes, hypertension and hypertriglyceridemia. Hepatic steatosis results from insulin resistance, which is the main pathophysiologic abnormality in the metabolic syndrome. The development of steatohepatitis requires both accumulation of fat and additional injurious processes in the liver which produce the steatohepatitis. The probability of having NAFLD rises with increasing body mass index (BMI) with over 80% of subjects having a BMI>35 having NAFLD. The development of steatohepatitis requires both accumulation of fat and additional injurious processes in the liver which produce the steatohepatitis. It is believed that oxidative stress plays an important role in this process.

On the other hand, fatty liver, which occurs after acute alcohol ingestion, is generally reversible with abstinence and is not believed to predispose to any chronic form of liver disease if abstinence or moderation is maintained. AFLD and ASH are forms of alcohol-induced liver injury that occurs with the consumption of a large quantity of alcohol over a prolonged period of time. These conditions encompass a spectrum of severity ranging from asymptomatic derangement of biochemistries to fulminant liver failure and death. Cirrhosis involves replacement of the normal hepatic parenchyma with extensive thick bands of fibrous tissue and regenerative nodules, which results in the clinical manifestations of portal hypertension and liver failure.

Since the gold-standard for diagnosis of the aforementioned liver conditions is a liver biopsy, hospital-based studies with liver biopsies are subject to ascertainment bias. Population based studies have utilized imaging modalities such as ultrasound and MRI to diagnose NAFLD/AFLD but are limited by the absence of histologic confirmation. Recently, changes in MRI have been correlated with hepatic lipid content enabling diagnosis of NAFLD/AFLD with relative high confidence. For example, based on MRI, it has been estimated that the overall prevalence of NAFLD in the United States is about 30%. However, MRI does not enable distinguishing between NAFL, NASH or ASH. Furthermore, the prevalence and incidence of NASH and ASH are not known because of the impossibility of performing liver biopsy in the general population.

NAFL is associated with a benign clinical course and the majority of cases of NAFL remain asymptomatic and free of fibrosis or development of steatohepatitis over a 5-10 year time frame from diagnosis. On the other hand, NASH can progress to cirrhosis in about 20% of cases and is considered as one of the major risk factors in developing hepatocellular carcinoma (HCC). The risk of cirrhosis is 30-40% in ASH patients who continue to drink alcohol. Natural histories of NASH and ASH patients are not completely defined yet.

About 15% of patients subject to liver transplantation have either NASH or cryptogenic cirrhosis, which is believed to be the end result of NASH as the underlying liver disease. NASH can be treated with bariatric surgery or with a variety of drugs such as insulin sensitizers. About 7.9% of the US population has persistently elevated liver enzymes with negative studies for viral hepatitis and other common causes of liver diseases that can be tested for with laboratory tests. Over 80% of such cases are felt to be due to NAFLD (NAFL or NASH). In those who have concomitant features of metabolic syndrome, the likelihood of NAFLD exceeds 90%. However, there are no non-invasive ways to distinguish NAFL from NASH.

Currently patients are offered a liver biopsy in order to diagnose NASH/ASH and to stage the level of inflammation and/or the fibrosis grade of the disease. It is important to note that a liver biopsy is invasive and painful and carries a small but definite risk of hemorrhage and death. Also, given the sheer number of subjects with NAFLD/AFLD, it is not logistically feasible to biopsy all subjects with NAFLD/AFLD. There is thus a great need for a simple, non-invasive method for diagnosis of NASH/ASH and liver lobular inflammation as well as monitoring of NAFLD/AFLD progression in this population.

Metabolic breath tests, utilized to assess the severity of liver disease, have been developed. Such tests are performed by administering a labelled compound either orally or intravenously. The compound is removed by the liver from the blood and metabolized, and a metabolic product is released back into the blood and excreted in the bile, urine, saliva or exhaled breath. Measuring the amount and/or rate of the metabolic product provides a measure of hepatic metabolic function.

Several compounds have been utilized to evaluate hepatic metabolic function in this manner, including indocyanine green, galactose, aminopyrine, caffeine, lidocaine, phenylalanine. Similarly other compounds have been proposed to evaluate mitochondrial function such as methionine and methacetin (AKA [N-(4-Methoxy-phenyl) acetamide] and sodium-octanoate).

Most of these methods have been abandoned due to impracticality or undesired side effects and/or limitations in performances associated with inter and intra patient variability.

Breath tests using ¹³C-labeled substrates provide a safe, non-invasive means for evaluating metabolism that is correlated with organ function. ¹³C is a stable, non-radioactive isotope, which has no known pharmacodynamic side effects, and which is released as ¹³CO₂ when the compound is metabolized by the target organ. The selected ¹³C-compound can be administered orally, is rapidly absorbed, exclusively metabolized by the targeted organ; and the ratio of ¹³CO₂/¹²CO₂ can be measured in exhaled breath within a short time (e.g. 20-30 minutes). The ability to detect, differentiate and quantify ¹³C and ¹²C in exhaled CO₂ has been greatly facilitated by the development of the BreathID® system, which allows assessment of an organ or hepatic impairment and other liver diseases.

Of particular interest for this invention are compounds that are metabolized in the mitochondrial compartment in hepatic cells through beta oxidation. It has been proposed in the art that beta-oxidation is impaired in fatty liver and non-alcoholic steatohepatitis and hepatocellular carcinoma. Accordingly, breath tests that are based on this phenomena have been proposed in patents and patent applications such as U.S. Pat. No. 8,512,258, U.S. Pat. No. 8,622,920, US Patent Application No. 2009/0131810 and US Patent Application No. 2011/61475264.

A variety of potential substances can be used to evaluate mitochondrial function and beta oxidation. One such compound is sodium octanoate (caprylic acid sodium salt, sodium caprylate, octanoic acid sodium, salt, sodium n-octanoate) which is metabolized through mitochondrial beta-oxidation in the liver.

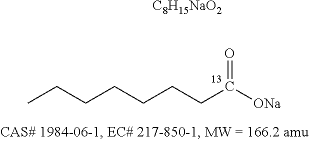

Octanoate is the salt form of octanoic (caprylic) acid having a water solubility of 50 mg/ml). Octanoate is a medium chain fatty acid that has physical and chemical properties rendering it a good candidate for assessing hepatic mitochondrial beta-oxidation in breath tests. This is due to the fact that octanoate is absorbed promptly from the intestinal lumen and transported rapidly to the liver, where it undergoes mitochondrial beta-oxidation. Subsequently, it is transformed into CO₂, which is exhaled and can be measured by a breath test.

¹³C-Octanaote Breath Test

It has been previously shown that NAFLD is associated with changes in fatty acid f3 oxidation which can impact ¹³CO₂ production. It was therefore hypothesized that upon evaluation of ¹³CO₂ after ingestion of ¹³C labeled octanoate distinction of NAFL patients from NASH patients may be enabled, and the progression of AFLD to ASH may be assessed as well.

However, irreproducible results due to inter and/or intra patient variability made the test result insufficiently accurate and of limited use. There thus remains a need for a non-invasive method enabling evaluation of NAFLD/AFLD or lobular inflammation and distinction between NAFL, NASH, AFL and ASH in an accurate and reproducible manner.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The following embodiments and aspects thereof are described and illustrated in conjunction with systems, tools and methods that are meant to be exemplary and illustrative, not limiting in scope. In various embodiments, one or more of the above-described problems have been reduced or eliminated, while other embodiments are directed to other advantages or improvements.

According to some embodiments, there is provided a method for evaluating a liver condition. The method comprises monitoring a metabolic product of a ¹³C labeled fatty acid in a subject's breath and normalizing the monitored metabolic product based on at least one characteristic of the patient.

According to some embodiments, the method(s) disclosed herein further includes distinguishing between NASH and NAFL. According to some embodiments, the method(s) disclosed herein further includes determining the level of NASH and/or NAFL. Each possibility is a separate embodiment.

According to some embodiments, the method(s) disclosed herein further includes distinguishing between ASH and AFL. According to some embodiments, the method(s) disclosed herein further includes determining the level of ASH and/or AFL. Each possibility is a separate embodiment.

According to some embodiments, the patient characteristics may include plasma glucose levels, HOMA score, HOMA IR, insulin and/or glucagon levels, plasma lipid levels, liver enzymes, coagulation tests, ammonia, bilirubin, inflammatory and/or immunological parameters (such as, but not limited to, cytokines or subsets of T lymphocytes), genetic data (including but not limited to genomics such as GWAS (genome wide associated studies in NASH, proteomics, metabolomics, lipid profiling, symptoms, clinical parameter(s), laboratory parameter(s) or any combination thereof. Each possibility is a separate embodiment.

According to some embodiments, measuring includes monitoring.

According to some embodiments, the term ‘monitor’ or ‘monitoring’ may refer to two of more measurements, for example, 2-5, 2-10, 5-30 measurements, periodic measurements, such as a measurement every 1-5 minutes, every 5-10 minutes, every 10-60 minutes or every 1-4 hours.

According to some embodiments, normalizing the measured metabolic product of the ¹³C labeled fatty acid may include applying an algorithm. For example, without being bound by any theory, high levels of glucose or insulin or low levels of glucagon may be associated with a decrease in beta oxidation, falsely indicating abnormal beta-oxidation function. Advantageously, the breath test disclosed herein enables assessing liver beta oxidation by normalizing the measured metabolic product of the ¹³C labeled fatty acid by taking into consideration the characteristics of the patient, here the glucose and/or insulin and/or glucagon levels of the subject.

According to some embodiments, evaluating the liver condition is further based on the subject's level of glucagon, level of ammonia, level of bilirubin, HOMA score, HOMA IR level of liver enzymes, inflammatory and/or immunological parameters (such as, but not limited to, cytokines or subsets of T lymphocytes), genetic data (including but not limited to genomics such as GWAS—genome wide associated studies) proteomics, metabolomics, lipid profiling, symptoms, clinical parameter(s), laboratory parameter(s), coagulation tests or any combination thereof.

According to some embodiments, the evaluation of the liver condition is further based on the subject's level of glucagon, level of ammonia, level of bilirubin, level of liver enzymes, inflammatory and/or immunological parameters (such as, but not limited to, cytokines or subsets of T lymphocytes), genetic data, (including but not limited to genomics such as GWAS—genome-wide associated studies) proteomics, metabolomics, lipid profiling, symptoms, clinical parameter(s), laboratory parameter(s), coagulation tests or any combination thereof.

According to some embodiments, the algorithm includes information about the etiology of the medical condition of the subject. It is understood by one of ordinary skill in the art that the impact of beta-oxidation may be different in a disease associated with a metabolic syndrome as opposed to the same disease induced by drugs.

According to some embodiments, the medical conditions may include, NAFLD, NAFL, NASH, AFLD, AFL, ASH, HCC or any other liver condition associated with changes in hepatic mitochondrial function.

According to some embodiments, the metabolic product is ¹³CO₂.

According to some embodiments, the method(s) disclosed herein further includes providing a treatment recommendation based on the normalized monitored metabolic product.

According to some embodiments, the method(s) disclosed herein may further include evaluating the risk of a patient with simple steatosis to develop NASH or ASH. According to some embodiments, the method(s) disclosed herein may further include evaluating the risk of NASH/ASH patients to deteriorate and develop fibrosis and/or cirrhosis. According to some embodiments, the method(s) disclosed herein may include predicting complications in patients with NASH/ASH cirrhosis. According to some embodiments, predicting complications in patients with NASH/ASH cirrhosis may include evaluating changes in the normalized metabolic product over time.

According to some embodiments, the method(s) disclosed herein may further include evaluating disease progression (improvement or deterioration) and/or a patient's response to treatment. According to some embodiments, evaluating the response to treatment may include monitoring the functional state of the liver, for example, when toxicity is suspected. According to some embodiments, the method(s) disclosed herein may include evaluating the recuperation of the liver after treatment. According to some embodiments, the method(s) disclosed herein may include assessing liver-mitochondrial function and, in turn, diagnosing the status, progression, treatment results, safety of treatment, prognosis of simple steatosis, NASH, NASH-cirrhosis, AFL, ASH or any combination thereof. Each possibility is a separate embodiment.

According to some embodiments, the ¹³C labeled fatty acid may include octanoate, alpha-keto-isocaproic acid (KICA), palmitic acid, any other fatty acid (whether saturated or unsaturated, natural and artificial) or any combination thereof. Each possibility is a separate embodiment. According to some embodiments, the ¹³C labeled fatty acid may include ¹³C-octanoate. According to some embodiments, the ¹³C labeled fatty acid may include phospholipids of any type such as, but not limited to, glycosphingolipids. It is understood that any other compound metabolized by the mitochondria (whether directly or indirectly), may also be used and, as such, fall within the scope of the present disclosure.

According to some embodiments, the ¹³C labeled fatty acid may be used in a combination with ¹³C labeled methacetin and/or methionine.

According to some embodiments, the ¹³C labeled fatty acid may be a combination of two or more ¹³C labeled fatty acids. According to some embodiments, the method(s) disclosed herein may include determining a ratio in the metabolism of the one or more ¹³C labeled fatty acids, as a measure of liver function.

According to some embodiments, the 13C labeled fatty acid(s) may be added in various dosages to detect and/or diagnose various medical conditions and diseases.

According to some embodiments, there is provided a method of evaluating a liver condition of a subject, the method includes: measuring, using one or more breath sensors, a metabolic product of a fatty acid, a salt or a derivative thereof, in the subject's breath after administering to the subject isotope labeled fatty acid, a salt or a derivative thereof, obtaining the subject's level of insulin, glucose, glucagon or any combination thereof and using a processing circuitry, evaluating the liver condition based on the subject's metabolic product of the fatty acid, salt or derivative thereof and the level of insulin, glucose, glucagon or any combination thereof.

According to some embodiments, there is provided a method of detecting and/or evaluating a liver inflammation in a subject, the method includes: measuring, using one or more breath sensors, a metabolic product of a fatty acid, a salt or a derivative thereof, in the subject's breath after administering to the subject an isotope labeled fatty acid, a salt or a derivative thereof and using a processing circuitry, detecting and/or evaluating a liver inflammation based on the subject's measured metabolic product of the fatty acid, salt or derivative thereof.

According to some embodiments, there is provided a method of evaluating Nonalcoholic Fatty Liver Disease (NAFLD) in a subject, the method includes: measuring, using one or more breath sensors, a metabolic product of a fatty acid, a salt or a derivative thereof, in the subject's breath after administering to the subject isotope labeled fatty acid, a salt or a derivative thereof and using a processing circuitry, evaluating the subject's NAFLD based on the metabolic product of the fatty acid, salt or derivative thereof.

According to some embodiments, there is provided a method of evaluating Alcoholic Fatty Liver Disease (AFLD) in a subject, the method includes: measuring, using one or more breath sensors, a metabolic product of a fatty acid, a salt or a derivative thereof, in the subject's breath after administering to the subject isotope labeled fatty acid, a salt or a derivative thereof and using a processing circuitry, evaluating the subject's AFLD based on the metabolic product of the fatty acid, salt or derivative thereof.

According to some embodiments, there is provided a method of evaluating a liver condition of a subject, the method includes: measuring, using one or more sensors, a metabolic product of an isotope labeled fatty acid, a salt or a derivative thereof, in a breath sample, measuring a level of insulin, glucose, glucagon or any combination thereof in a sample of blood, urine, plasma and/or intercellular fluid, and using a processing circuitry, evaluating the liver condition based on the subject's metabolic product of the fatty acid, salt or derivative thereof and the level of insulin, glucose, glucagon or any combination thereof.

According to some embodiments, there is provided a method of detecting and/or evaluating a liver inflammation of a subject, the method includes: measuring, using one or more sensors, a metabolic product of an isotope labeled fatty acid, a salt or a derivative thereof, in a breath sample and using a processing circuitry, detecting and/or evaluating the liver inflammation based on the subject's metabolic product of the fatty acid, salt or derivative thereof.

According to some embodiments, there is provided a method of evaluating Nonalcoholic Fatty Liver Disease (NAFLD) of a subject, the method includes: measuring, using one or more sensors, a metabolic product of an isotope labeled fatty acid, a salt or a derivative thereof, in a breath sample and using a processing circuitry, evaluating the subject's NAFLD based on the subject's metabolic product of the fatty acid, salt or derivative thereof.

According to some embodiments, there is provided a method of evaluating Alcoholic Fatty Liver Disease (AFLD) of a subject, the method includes: measuring, using one or more sensors, a metabolic product of an isotope labeled fatty acid, a salt or a derivative thereof, in a breath sample and using a processing circuitry, evaluating the subject's AFLD based on the subject's metabolic product of the fatty acid, salt or derivative thereof.

According to some embodiments, the term “liver disease” as used herein, may refer to an acute or chronic condition. According to some embodiments, the term “lobular inflammation” as used herein, may refer to an acute or chronic condition.

According to some embodiments, the labeled fatty acid, its salt or derivative thereof may include a saturated, unsaturated, natural, artificial labeled fatty acid, salts or derivatives thereof or any combination thereof.

According to some embodiments, the labeled fatty acid, its salt or derivative thereof may include octanoic acid, alpha-keto-isocaproic acid (KICA), palmitic acid and phospholipids, salts or derivatives thereof or any combination thereof.

According to some embodiments, the labeled fatty acid, its salt or derivative thereof may include labeled octanoic acid, a salt or a derivative thereof.

According to some embodiments, evaluating the liver condition/liver inflammation may include evaluating the level of nonalcoholic fatty liver (NAFL), non-alcoholic steatohepatitis (NASH), alcoholic fatty liver (AFL) and/or alcoholic steatohepatitis (ASH) conditions in the subject. According to some embodiments, evaluating NAFLD may include evaluating the level of nonalcoholic fatty liver (NAFL) and/or non-alcoholic steatohepatitis (NASH) conditions in the subject. According to some embodiments, evaluating the liver AFLD may include evaluating the level of alcoholic fatty liver (AFL) and/or alcoholic steatohepatitis (ASH) conditions in the subject.

According to some embodiments, evaluating the liver condition, inflammation or NAFLD may include distinguishing between nonalcoholic fatty liver (NAFL) and non-alcoholic steatohepatitis (NASH) conditions in the subject.

According to some embodiments, evaluating the liver condition, inflammation or AFLD may include distinguishing between alcoholic fatty liver (AFL) and alcoholic steatohepatitis (ASH) conditions in the subject.

According to some embodiments, evaluating the liver condition, NAFLD/AFLD may include detecting and/or evaluating the level of liver inflammation in the subject.

According to some embodiments, evaluating the liver condition, inflammation, NAFLD/AFLD may include comparing any measured value, using a processor/a processing circuitry to reference value(s). Reference value(s) may be predetermined reference value(s) taken, for example, from literature, databases and the like.

According to some embodiments, the method(s) disclosed herein may further include measuring the subject's level of insulin, glucose, glucagon or any combination thereof during the day of measuring of the subject's breath. According to some embodiments, the measurements of the subject's level of insulin, glucose, glucagon or any combination may be performed within 0-15 minutes, 15-30 minutes, 30-45 minutes, 45-60 minutes, 60-90 minutes, 90-120 minutes, 2-3 hours, 3-4 hours, 4-5 hours, 5-6 hours, 6-8 hours, 8-10 hours, 10-12 hours, 12-16 hours, 16-20 hours or 20-24 hours prior to the breath test. According to some embodiments, the measurements of the subject's level of insulin, glucose, glucagon or any combination may be performed within 0-15 minutes, 15-30 minutes, 30-45 minutes, 45-60 minutes, 60-90 minutes, 90-120 minutes, 2-3 hours, 3-4 hours, 4-5 hours, 5-6 hours, 6-8 hours, 8-10 hours, 10-12 hours, 12-16 hours, 16-20 hours or 20-24 hours after to the breath test. After to the breath test may mean after the completion of the breath test or after the beginning of the breath test.

According to some embodiments, measuring the subject's level of insulin, glucose, glucagon or any combination thereof may be performed during the measuring of the subject's breath.

According to some embodiments, the subject's level of insulin, glucose, glucagon or any combination thereof may be measured in the blood. According to some embodiments, the subject's level of glucose may be measured in the urine. According to some embodiments, the subject's level of glucose may be measured in the plasma. According to some embodiments, the subject's level of glucose may be measured in the intercellular fluid. According to some embodiments, the subject's level of glucose may be measured trans-dermally. According to some embodiments, the subject's level of glucose may be measured sub-cutaneously.

According to some embodiments, the method(s) disclosed herein may further include measuring a metabolic product of methacetin or a derivative thereof, in a subject's breath after administering to the subject isotope labeled methacetin or a derivative thereof and wherein the evaluation of the liver condition/liver inflammation may further be based on the subject's metabolic product of the methacetin or derivative thereof.

According to some embodiments, the method(s) disclosed herein may further include measuring a metabolic product of methacetin or a derivative thereof, in a subject's breath after administering to the subject isotope labeled methacetin or a derivative thereof and wherein the evaluation of NAFLD/AFLD may further be based on the subject's metabolic product of the methacetin or derivative thereof.

According to some embodiments, the method(s) disclosed herein may further include measuring a metabolic product of methionine or a derivative thereof, in a subject's breath after administering to the subject isotope labeled methionine or a derivative thereof and wherein the evaluation of the liver condition/liver inflammation may further be based on the subject's metabolic product of the methionine or derivative thereof.

According to some embodiments, the method(s) disclosed herein may further include measuring a metabolic product of methionine or a derivative thereof, in a subject's breath after administering to the subject isotope labeled methionine or a derivative thereof and wherein the evaluation of the NAFLD/AFLD may further be based on the subject's metabolic product of the methionine or derivative thereof.

According to some embodiments, the method(s) disclosed herein may further include measuring an isotope level of a metabolic product of methacetin or a derivative thereof, in a subject's breath sample following administration of isotope labeled methacetin or a derivative thereof and wherein the evaluation of the liver condition/liver inflammation may further be based on the subject's isotope level of the metabolic product of the methacetin or derivative thereof.

According to some embodiments, the method(s) disclosed herein may further include measuring an isotope level of a metabolic product of methacetin or a derivative thereof, in a subject's breath sample following administration of isotope labeled methacetin or a derivative thereof and wherein the evaluation of the NAFLD/AFLD may further be based on the subject's isotope level of the metabolic product of the methacetin or derivative thereof.

According to some embodiments, the method(s) disclosed herein may further include measuring an isotope level of a metabolic product of methionine or a derivative thereof, in a subject's breath sample following administration of isotope labeled methionine or a derivative thereof and wherein the evaluation of the liver condition/liver inflammation may further be based on the subject's isotope level of the metabolic product of the methionine or derivative thereof.

According to some embodiments, the method(s) disclosed herein may further include measuring an isotope level of a metabolic product of methionine or a derivative thereof, in a subject's breath sample following administration of isotope labeled methionine or a derivative thereof and wherein the evaluation of the NAFLD/AFLD may further be based on the subject's isotope level of the metabolic product of the methionine or derivative thereof.

According to some embodiments, evaluating the liver condition, NAFLD/AFLD may further be based on the subject's level of ammonia, level of bilirubin, level of liver enzymes, alcohol drinking habits, inflammatory and/or immunological parameters, genetic data, proteomics, metabolomics, lipid profiling, symptoms, clinical parameters, laboratory parameters, coagulation tests or any combination thereof.

According to some embodiments, the evaluation of liver condition/liver inflammation may further be based on a physiological and/or medical parameter such as age, gender, weight, height, waist circumference, blood related parameter, body mass index (BMI), and medication therapy related parameter.

According to some embodiments, the evaluation of NAFLD/AFLD may further be based on a physiological and/or medical parameter such as age, gender, weight, height, waist circumference, blood related parameter, body mass index (BMI), and medication therapy related parameter.

According to some embodiments, the measurement may include monitoring. According to some embodiments, the measurement may include an on-line monitoring. According to some embodiments, the measurement may include a continuous monitoring. According to some embodiments, the monitoring may be a real-time monitoring. According to some embodiments, the monitoring may be performed after breathing out (i.e., exhaling), in a breath sample, previously obtained from a subject.

According to some embodiments, the metabolic product may be CO₂.

According to some embodiments, isotope labeled fatty acid may include fatty acids labeled with carbon-13, carbon-14, oxygen-18 or any combination thereof.

According to some embodiments, the liver condition may include a liver related disease, inflammation, malfunction, injury, transplantation, abnormality, fat accumulation, increased metabolism, decreased metabolism or a combination thereof.

According to some embodiments, the detection/evaluation of the liver inflammation may include assigning a 0-3 score according to NAS for liver lobular inflammation.

According to some embodiments, detecting and/or evaluating a liver inflammation may be performed on subjects suffering from nonalcoholic fatty liver disease (NAFLD).

According to some embodiments, detecting and/or evaluating a liver inflammation may be performed on subjects suffering from alcoholic fatty liver disease (AFLD).

According to some embodiments, the subject is not suffering from cirrhosis.

According to some embodiments, there is provided a device for evaluating a liver condition of a subject, the device includes: one or more breath sensors adapted to measure an isotope level of a metabolic product of labeled fatty acid, or a salt or a derivative thereof in the subject's breath and a processing circuitry adapted to sample measurements of the one or more sensors and evaluate the liver condition of the subject based on the measured isotope level and on the subject's level of insulin, glucose, glucagon or any combination thereof.

According to some embodiments, there is provided a device for detecting and/or evaluating a liver inflammation in a subject, the device includes: one or more breath sensors adapted to measure an isotope level of a metabolic product of labeled fatty acid, or a salt or a derivative thereof in the subject's breath and a processing circuitry adapted to sample measurements of the one or more sensors and detect and/or evaluate the liver inflammation of the subject based on the measured isotope level of the metabolic product of the labeled fatty acid, or a salt or a derivative thereof.

According to some embodiments, there is provided a device for evaluating Nonalcoholic Fatty Liver Disease (NAFLD) in a subject, the device includes: one or more breath sensors adapted to measure an isotope level of a metabolic product of labeled fatty acid, or a salt or a derivative thereof in the subject's breath and a processing circuitry adapted to sample measurements of the one or more sensors and evaluate NAFLD in the subject based on the measured isotope level.

According to some embodiments, there is provided a device for evaluating Alcoholic Fatty Liver Disease (AFLD) in a subject, the device includes: one or more breath sensors adapted to measure an isotope level of a metabolic product of labeled fatty acid, or a salt or a derivative thereof in the subject's breath and a processing circuitry adapted to sample measurements of the one or more sensors and evaluate AFLD in the subject based on the measured isotope level.

According to some embodiments, there is provided a device for evaluating a liver condition of a subject, the device includes: one or more sensors adapted to measure an isotope level of a metabolic product of labeled fatty acid, or a salt or a derivative thereof in the subject's breath sample and a processing circuitry adapted to sample measurements of the one or more sensors and evaluate the liver condition of the subject based on the measured isotope level and on the level of insulin, glucose, glucagon or any combination thereof measured in a sample of blood, urine, plasma and/or intercellular fluid.

According to some embodiments, there is provided a device for detecting and/or evaluating a liver inflammation of a subject, the device includes: one or more sensors adapted to measure an isotope level of a metabolic product of labeled fatty acid, or a salt or a derivative thereof in the subject's breath sample and a processing circuitry adapted to sample measurements of the one or more sensors and detect and/or evaluate the liver inflammation of the subject based on the measured isotope level and on the level of insulin, glucose, glucagon or any combination thereof measured in a sample of blood, urine, plasma and/or intercellular fluid.

According to some embodiments, there is provided a device for evaluating Nonalcoholic Fatty Liver Disease (NAFLD) of a subject, the device includes: one or more sensors adapted to measure an isotope level of a metabolic product of labeled fatty acid, or a salt or a derivative thereof in the subject's breath sample and a processing circuitry adapted to sample measurements of the one or more sensors and evaluate the NAFLD of the subject based on the measured isotope level and on the level of insulin, glucose, glucagon or any combination thereof measured in a sample of blood, urine, plasma and/or intercellular fluid.

According to some embodiments, there is provided a device for evaluating Alcoholic Fatty Liver Disease (AFLD) of a subject, the device includes: one or more sensors adapted to measure an isotope level of a metabolic product of labeled fatty acid, or a salt or a derivative thereof in the subject's breath sample and a processing circuitry adapted to sample measurements of the one or more sensors and evaluate the AFLD of the subject based on the measured isotope level and on the level of insulin, glucose, glucagon or any combination thereof measured in a sample of blood, urine, plasma and/or intercellular fluid.

According to some embodiments, the processing circuitry may be configured to sample the measurements at a continuous mode.

According to some embodiments, the labeled fatty acid, its salt or derivative thereof may include a saturated, unsaturated, natural, artificial labeled fatty acid, salts or derivatives thereof or any combination thereof.

According to some embodiments, the labeled fatty acid, its salt or derivative thereof may include octanoic acid, alpha-keto-isocaproic acid (KICA), palmitic acid and phospholipids, salts or derivatives thereof or any combination thereof.

According to some embodiments, the labeled fatty acid, its salt or derivative thereof may include labeled octanoic acid, a salt or a derivative thereof.

According to some embodiments, evaluating the liver condition/liver inflammation may include evaluating the level of nonalcoholic fatty liver (NAFL) and/or non-alcoholic steatohepatitis (NASH) conditions in a subject.

According to some embodiments, evaluating the liver condition/liver inflammation may include evaluating the level of alcoholic fatty liver (AFL) and/or alcoholic steatohepatitis (ASH) conditions in a subject.

According to some embodiments, evaluating the liver condition/liver inflammation may include distinguishing between nonalcoholic fatty liver (NAFL) and non-alcoholic steatohepatitis (NASH) conditions in a subject.

According to some embodiments, evaluating the liver condition/liver inflammation may include distinguishing between alcoholic fatty liver (AFL) and alcoholic steatohepatitis (ASH) conditions in a subject.

According to some embodiments, evaluating the liver condition, NAFLD/AFLD may include detecting and/or evaluating the level of liver inflammation in a subject.

According to some embodiments, the one or more breath sensors may further be configured to measure an isotope level of a metabolic product of a methacetin or a derivative thereof in the subject's breath after administering to the subject isotope labeled methacetin or a derivative thereof, and wherein the evaluation of the liver condition/liver inflammation may further be based on the subject's measured isotope level of the metabolic product of the labeled methacetin or derivative thereof.

According to some embodiments, the one or more breath sensors may further be configured to measure an isotope level of a metabolic product of a methacetin or a derivative thereof in the subject's breath after administering to the subject isotope labeled methacetin or a derivative thereof, and wherein the evaluation of NAFLD/AFLD may further be based on the subject's measured isotope level of the metabolic product of the labeled methacetin or derivative thereof.

According to some embodiments, the one or more breath sensors may further be configured to measure an isotope level of a metabolic product of a methionine or a derivative thereof in the subject's breath after administering to the subject isotope labeled methionine or a derivative thereof, and wherein the evaluation of the liver condition/liver inflammation may further be based on the subject's measured isotope level of the metabolic product of the labeled methionine or derivative thereof.

According to some embodiments, the one or more breath sensors may further be configured to measure an isotope level of a metabolic product of a methionine or a derivative thereof in the subject's breath after administering to the subject isotope labeled methionine or a derivative thereof, and wherein the evaluation of NAFLD/AFLD may further be based on the subject's measured isotope level of the metabolic product of the labeled methionine or derivative thereof.

According to some embodiments, the evaluation of the liver condition, NAFLD/AFLD may further be based on the subject's level of ammonia, level of bilirubin, level of liver enzymes, alcohol drinking habits, inflammatory and/or immunological parameters, genetic data, proteomics, metabolomics, lipid profiling, symptoms, clinical parameters, laboratory parameters, coagulation tests or any combination thereof.

According to some embodiments, the evaluation of liver condition/liver inflammation may further be based on the subject's physiological and/or medical parameter including age, gender, weight, height, waist circumference, blood related parameter, body mass index (BMI) and medication therapy related parameter.

According to some embodiments, the evaluation of liver condition/liver inflammation may further be based on the subject's physiological and/or medical parameter including age, gender, weight, height, waist circumference, blood related parameter, body mass index (BMI) and medication therapy related parameter. According to some embodiments the evaluation of NAFLD AFLD may further be based on the subject's physiological and/or medical parameter including age, gender, weight, height, waist circumference, blood related parameter, body mass index (BMI) and medication therapy related parameter.

According to some embodiments, the measuring may include monitoring. According to some embodiments the measuring may include an on-line monitoring. According to some embodiments the measuring may include a continuous monitoring. According to some embodiments, the monitoring may be a real-time monitoring. According to some embodiments, the monitoring may be performed after breathing out (i.e., exhaling), in a breath sample, previously obtained from a subject.

According to some embodiments, the metabolic product may be CO₂.

According to some embodiments isotope labeled fatty acid may include fatty acids labeled with carbon-13, carbon-14, oxygen-18 or any combination thereof.

According to some embodiments, the liver condition may include a liver related disease, inflammation, malfunction, injury, transplantation, abnormality, fat accumulation, increased metabolism, decreased metabolism or a combination thereof.

According to some embodiments, the detection/evaluation of the liver inflammation may include assigning a 0-3 score according to NAS for liver lobular inflammation.

According to some embodiments, detecting and/or evaluating a liver inflammation is performed on subjects suffering from nonalcoholic fatty liver disease (NAFLD).

According to some embodiments, detecting and/or evaluating a liver inflammation is performed on subjects suffering from alcoholic fatty liver disease (AFLD).

According to some embodiments the subject is not suffering from cirrhosis.

According to some embodiments, the one or more breath sensors may further be configured to measure an isotope level of a metabolic product of methacetin or a derivative thereof, in a subject's breath sample following administration of isotope labeled methacetin or a derivative thereof and wherein the evaluation of the liver condition/liver inflammation may further be based on the subject's isotope level of the metabolic product of the methacetin or derivative thereof.

According to some embodiments, the one or more breath sensors may further be configured to measure an isotope level of a metabolic product of methacetine or a derivative thereof, in a subject's breath sample following administration of isotope labeled methacetine or a derivative thereof and wherein the evaluation of the NAFLD/AFLD may further be based on the subject's isotope level of the metabolic product of the methacetin or derivative thereof.

According to some embodiments, the one or more breath sensors may further be configured to measure an isotope level of a metabolic product of methionine or a derivative thereof, in a subject's breath sample following administration of isotope labeled methionine or a derivative thereof and wherein the evaluation of the liver condition/liver inflammation may further be based on the subject's isotope level of the metabolic product of the methionine or derivative thereof.

According to some embodiments, the one or more breath sensors may further be configured to measure an isotope level of a metabolic product of methionine or a derivative thereof, in a subject's breath sample following administration of isotope labeled methionine or a derivative thereof and wherein the evaluation of the NAFLD/AFLD may further be based on the subject's isotope level of the metabolic product of the methionine or derivative thereof.

According to some embodiments the measurement may include monitoring. According to some embodiments the measurement may include an on-line monitoring. According to some embodiments the measurement may include a continuous monitoring. According to some embodiments, the monitoring may be a real-time monitoring. According to some embodiments, the monitoring may be performed after breathing out (i.e., exhaling), in a breath sample, previously obtained from a subject.

More details and features of the current invention and its embodiments may be found in the description and the attached drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE FIGURES

Exemplary embodiments are illustrated in the referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below:

FIG. 1 depicts ROC curve of ¹³C-octanoate breath test percentage dose recovery (PDR) peak values in severe vs. non-severe Nonalcoholic Fatty Liver Disease (NAFLD) patients, according to some embodiments;

FIG. 2 depicts ROC curve of ¹³C-octanoate breath test PDR peak values modified according to blood glucose and insulin values in severe vs. non-severe NAFLD patients, according to some embodiments;

FIG. 3 depicts ROC curve of ¹³C-octanoate breath test PDR peak values in NAFLD patients suffering from liver lobular inflammation vs. NAFLD patients not suffering from liver lobular inflammation, according to some embodiments; and

FIG. 4 depicts a boxplot diagram of ¹³C-octanoate breath test PDR peak values in healthy subjects not suffering from NAFLD or lobular inflammation vs. NAFLD patients not suffering from lobular inflammation vs. NAFLD subjects suffering from stage 1 lobular inflammation vs. NAFLD subjects suffering from stage 2 lobular inflammation, according to some embodiments.

### DETAILED DESCRIPTION OF THE INVENTION

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

In the description and claims of the application, each of the words “comprise” “include” and “have”, and forms thereof, are not necessarily limited to members in a list with which the words may be associated.

### Examples

### Example 1: Octanoate Breath Test (OBT) for Detection of NAFLD

The study group included 26 subjects (18 females and 8 males) suffering from NAFLD. The population was divided into severe and non-severe NAFLD patients based on the NAS and the fibrosis score, as an example for a standard histology based score for disease severity. NAS greater than 4 and/or fibrosis≧1c was considered severe. According to the aforementioned considerations, 14 subjects were classified as suffering from severe disease, whereas 12 subjects were considered as suffering from non-severe nonalcoholic fatty liver disease.

First, subjects underwent dynamic ¹³C-octanoate breath test (OBT) using BreathID® device (Exalenz Bioscience Ltd.).

The breath tests were performed according to the following procedure:

a. Preparation of the Study Subject:

Subjects were asked to perform the breath test after an overnight fast (including morning medication). The subjects were allowed to drink small amounts of water until 1 hour prior to test. The subjects rested for 3-5 minutes prior to the test start (to assure that breathing rate and pulse are normal and constant throughout the test).

b. Preparation of ¹³C-Octanaote:

100 mg of ¹³C-Octanoate powder were emptied into a disposable cup and 150 cc of water were added. The mixture was mixed until the substrate had been completely dissolved.

Just prior to the examination, this solution was poured into a disposable cup.

c. Administration of the Breath Test:
i. Each patient was asked to sit in a chair in the room where the test was performed.
ii. A nasal cannula was attached to a BreathID® device and to the patient.
iii. The BreathID® device was activated and collected the patient's baseline exhaled CO₂ for approximately 2 minutes.
iv. The patient was then instructed by the medical staff and by an indication on the device to drink the test substrate.
v. The patient remained seated in the chair, breathing in a normal manner for the next 60 minutes.
vi. The BreathID® device continuously measured and analyzed the patient's exhaled breath in real time. As the test substrate was metabolized, the value of the ¹³CO₂/¹²CO₂ ratio changed and was calculated in real time by the BreathID® system from the exhaled breath. The BreathID® also calculated in real time the percentage dose recovery (PDR), expressed in %/hour. This value was displayed on the screen of the BreathID® device as it is calculated in real time.
vii. If at any time the device did not detect a patient's breath, or if there was any other deviation from the desired test requirements, the device produced an appropriate warning signal.
viii. At the completion of the procedure, the nasal cannula was removed and the patient was allowed to leave the testing room.

The patient was under the supervision of the physician or any other qualified medical staff during the entire test.

For each breath test, a percentage dose recovery (PDR) curve was generated. The octanoate breath test PDR peak values were grouped according to severe/non-severe NAFLD, as in the specifications hereinabove.

Two experiments were performed: In experiment A, the predictive value of the OBT PDR peak in discriminating between severe and non-severe NAFLD patients was assessed without incorporating glucose and insulin level parameters. In experiment B, the predictive value of the OBT PDR peak modified by incorporation of glucose and insulin level parameters, in discriminating between severe and non-severe NAFLD patients was assessed.

Experiment A: prediction of NAFLD severity according to OBT PDR peak

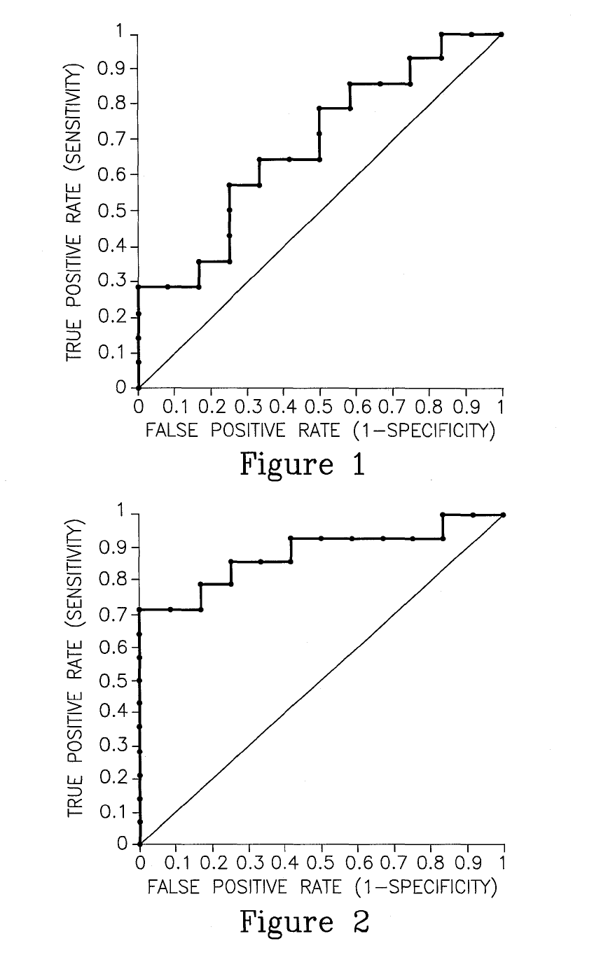

A Receiver Operating Characteristic (ROC) curve was generated (FIG. 1) and its Area Under the Curve, AUC_{ROC}, was calculated in order to assess the predictive value of the PDR peak in discriminating between severe and non-severe NAFLD patients.

An AUC_{ROC} value of 0.68 (p=0.0426) was obtained, showing that discrimination between severe and non-severe NAFLD can be obtained when analyzing the OBT PDR dataset with no consideration of blood glucose and insulin levels in patients.

Experiment B: prediction of NAFLD severity according to an adjusted OBT PDR peak modified according to glucose and insulin levels.

Blood samples were collected from the 26 subjects on the day of the octanoate breath test and their blood glucose and insulin were measured.

A regression model that uses the collected blood insulin and glucose levels to normalize the measured OBT PDR peak for the modified level of beta-oxidation was developed, producing a modified PDR peak. The specifications of the algorithm are presented in Table 1. Other algorithms may be developed for the same or other disease severity scores.

TABLE 1
Adjusted PDR Peak = x₀ + x₁ * PDR Peak + x₂ * Glucose
levels + x₃ * Insulin levels

| | Severe | | | | Class. Value |
|---|---|---|---|---|---|
| p-value | Wald | Std. Err | Coef. | Var. | Pred. Att. |
| — | — | — | =2.710 | x₀ | Constant |
| 0.0122 | 6.281 | 0.1391 | =−0.35 | x₁ | PDRPeak |
| 0.0201 | 5.4 | 0.01855 | =0.04 | x₂ | Glucose |
| 0.3488 | 0.8779 | 0.02897 | =0.03 | x₃ | Insulin |

A second Receiver Operating Characteristic (ROC) curve was generated (FIG. 2), and AUC_{ROC} was calculated in order to assess the predictive value of the modified PDR peak in discriminating between severe and non-severe NAFLD patients.

A much improved ROC curve was obtained compared with the preliminary ROC curve which ignored the influence of blood glucose and insulin levels (Experiment 1). The AUC_{ROC} value of the curve was 0.88 (p<0.0001), meaning that very high discrimination between severe and non-severe NAFLD can be obtained when analyzing the OBT PDR dataset with consideration of the subjects' blood glucose and insulin levels. This observation suggests that for diagnostic purposes these parameters should be included in addition to the OBT PDR peak in differentiating NAFLD in patients between severe and not severe.

### Example 2: Octanoate Breath Test (OBT) for Detection and Evaluation of Liver Lobular Inflammation

Forty-nine (49) human subjects suffering from NAFLD (33 females and 16 males) were investigated. The population was divided into subjects suffering from liver lobular inflammation and those not suffering from liver lobular inflammation. Forty-five (45) subjects were classified as suffering, whereas 4 subjects were considered as not suffering, from lobular inflammation, as previously assessed in liver histology.

First, the subjects underwent dynamic ¹³C-octanoate breath test (OBT) using BreathID® device (Exalenz Bioscience Ltd.), according to the procedures for preparation of the study subject, preparation of ¹³C-octanaote and administration of the breath test described in Example 1.

For each breath test, a percentage dose recovery (PDR) curve was generated. The Octanoate Breath Test PDR peak values were grouped according to the presence of lobular inflammation in the subject's liver, predetermined according to the specifications hereinabove.

Two experiments were performed: In experiment A, the predictive value of the OBT PDR peak in discriminating between patients suffering from liver lobular inflammation and those not suffering from liver lobular inflammation was assessed; In experiment B, an assessment of the ability of the OBT PDR peak in evaluating different levels of inflammation was made.

Experiment A: detection of liver lobular inflammation according to OBT PDR peak.

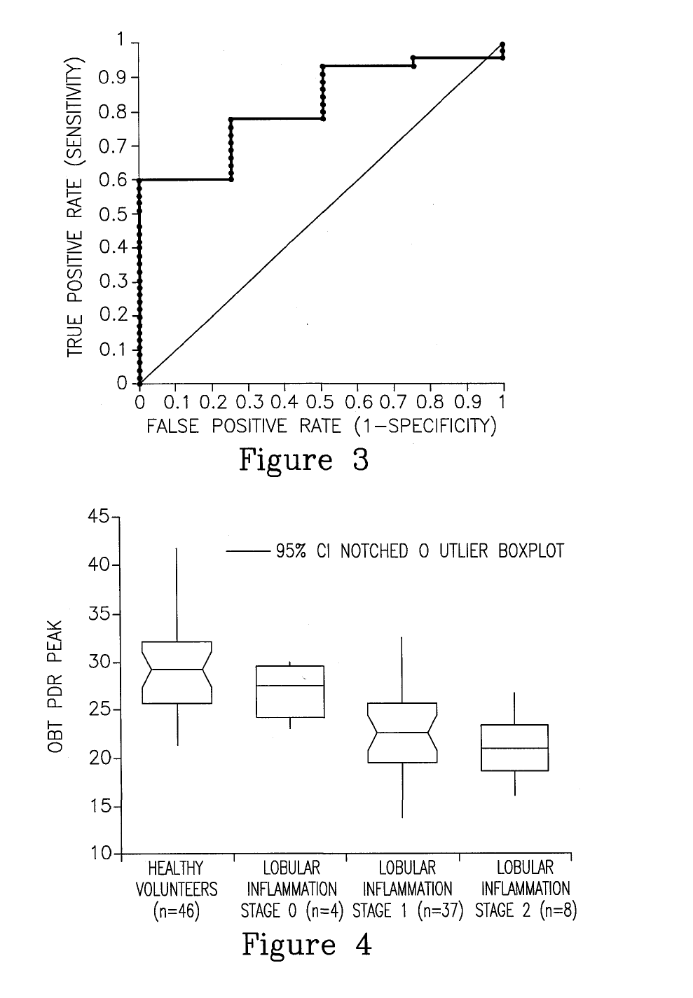

A Receiver Operating Characteristic (ROC) curve was generated (FIG. 3) and AUC_{ROC} was calculated in order to assess the predictive value of the PDR peak in discriminating between NAFLD patients suffering from any grade of lobular inflammation and NAFLD patients not suffering from liver lobular inflammation.

An AUC_{ROC} value of 0.82 (p=0.0003) was obtained, meaning that indeed the octanoate breath test PDR peak is a very good measure of assessment for presence of any grade of lobular inflammation in the subjects and differentiating the population of nonalcoholic fatty liver disease patients who suffer from liver lobular inflammation and the population of said disease patients who do not suffer from liver lobular inflammation. (FIG. 3)

Experiment B: evaluation of liver lobular inflammation according to OBT PDR peak.

In this experiment, presented in FIG. 4, 49 human subjects suffering from NAFLD (33 females and 16 males) and 46 healthy subjects (23 females and 23 males) were investigated. The 49-subject population was divided into three groups according to the lobular inflammation in the NAS score: 0 (no inflammation), 1 (<2 per 20× field) and 2 (2-4 per 20× field). The 46 healthy subjects, although not biopsied, were considered as having no lobular inflammation. Four (4) of the NAFLD subjects were considered not suffering from lobular inflammation according to biopsy, and therefore were assigned “0” in lobular inflammation according to the NAS score. The remaining 45 NAFLD patients, showing presence of lobular inflammation, was divided into 37 subjects assigned “1” in lobular inflammation according to the NAS score and 8 subjects assigned “2” in lobular inflammation according to the NAS score, as previously assessed in liver histology.

All subjects underwent ¹³C-OBT using BreathID® device (Exalenz Bioscience Ltd.) according to the procedures described in Example 1. For each breath test, a percentage dose recovery (PDR) curve was generated.

The Octanoate Breath Test PDR peak values were grouped according to the level of lobular inflammation in the subject's liver, predetermined according to the specifications hereinabove (healthy/no inflammation/stage 1 inflammation/stage 2 inflammation).

A boxplot was generated plotting the OBT PDR values versus inflammation severity. The study group was divided into four groups: healthy subjects, not suffering from NAFLD or lobular inflammation (n=46); Nonalcoholic fatty liver disease patients not suffering from lobular inflammation (n=4); NAFLD subjects suffering from stage 1 lobular inflammation according to NAS score (n=37); NAFLD subjects suffering from stage 2 lobular inflammation according to NAS score (n=8). The range of PDR values of the entire population is represented by vertical lines. Quartiles of PDR peak values are represented in the four box plots for each group separately. Horizontal lines (bands) inside the boxes represent the median value of PDR peaks in each group.

The results are shown in FIG. 4. It can be seen in the Figure that there is very little overlap between the combined population which encompass healthy subjects (not suffering from NAFLD) and subjects assigned 0 (suffering from NAFLD, but not from lobular inflammation) versus the combined population of subjects assigned lobular inflammation stages of 1 and 2. Furthermore, there is a high overlap between the healthy volunteers deemed to be inflammation free and the NAFLD patients assigned “0”. In addition, there is a moderate trend for a decrease in OBT PDR values upon increasing severity of the inflammation, as can be witnessed from comparing median values of stage 1 liver lobular inflammation patients (median=22.63) versus stage 2 liver lobular inflammation patients (median=20.89).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1.-147. (canceled)

148. A device for evaluating a liver condition of a subject, the device comprising:
one or more breath sensors adapted to measure an isotope level of a metabolic product of labeled fatty acid, or a salt or a derivative thereof in the subject's breath; and
a processing circuitry adapted to sample measurements of said one or more sensors and evaluate the liver condition of the subject based on the measured isotope level and on the subject's level of insulin, glucose, glucagon or any combination thereof.

149. The device of claim 148, wherein said processing circuitry is configured to sample the measurements at a continuous mode.

150. The device of claim 148, wherein evaluating the liver condition comprises evaluating the level of nonalcoholic fatty liver (NAFL) and/or non-alcoholic steatohepatitis (NASH) conditions in a subject.

151. The device of claim 148, wherein evaluating the liver condition comprises evaluating the level of alcoholic fatty liver (AFL) and/or alcoholic steatohepatitis (ASH) conditions in a subject.

152. The device of claim 148, wherein evaluating the liver condition comprises distinguishing between nonalcoholic fatty liver (NAFL) and non-alcoholic steatohepatitis (NASH) conditions in a subject.

153. The device of claim 148, wherein evaluating the liver condition comprises distinguishing between alcoholic fatty liver (AFL) and alcoholic steatohepatitis (ASH) conditions in a subject.

154. The device of claim 148, wherein said one or more breath sensors are further configured to measure an isotope level of a metabolic product of a methacetin or a derivative thereof in the subject's breath after administering to the subject isotope labeled methacetin or a derivative thereof, and wherein the evaluation of the liver condition is further based on the subject's measured isotope level of the metabolic product of the labeled methacetin or derivative thereof.

155. The device of claim 148, wherein said one or more breath sensors are further configured to measure an isotope level of a metabolic product of a methionine or a derivative thereof in the subject's breath after administering to the subject isotope labeled methionine or a derivative thereof, and wherein the evaluation of the liver condition is further based on the subject's measured isotope level of the metabolic product of the labeled methionine or derivative thereof.

156. A method of detecting and/or evaluating a liver inflammation in a subject, the method comprising:
measuring, using one or more breath sensors, a metabolic product of a fatty acid, a salt or a derivative thereof, in the subject's breath after administering to the subject an isotope labeled fatty acid, a salt or a derivative thereof;
using a processing circuitry, detecting and/or evaluating a liver inflammation based on the subject's measured metabolic product of the fatty acid, salt or derivative thereof.

157. The method of claim 156, wherein said labeled fatty acid, said salt or said derivative thereof comprises a saturated, unsaturated, natural, artificial labeled fatty acid, salts or derivatives thereof or any combination thereof.

158. The method of claim 156, wherein said labeled fatty acid, said salt or said derivative thereof is selected from a group consisting of: octanoic acid, alpha-keto-isocaproic acid (KICA), palmitic acid, phospholipids, salts or derivatives thereof or any combination thereof.

159. The method of claim 156, wherein said labeled fatty acid, said salt or said derivative thereof comprises labeled octanoic acid, a salt or a derivative thereof.

160. The method of claim 156, further comprising evaluating the level of nonalcoholic fatty liver (NAFL) and/or non-alcoholic steatohepatitis (NASH) conditions in the subject.

161. The method of claim 156, further comprising evaluating the level of alcoholic fatty liver (AFL) and/or alcoholic steatohepatitis (ASH) conditions in the subject.

162. The method of claim 156, further comprising distinguishing between a nonalcoholic fatty liver (NAFL) and non-alcoholic steatohepatitis (NASH) conditions in the subject.

163. The method of claim 156, further comprising distinguishing between an alcoholic fatty liver (AFL) and alcoholic steatohepatitis (ASH) condition in the subject.

164. The method of claim 156, further comprising measuring a metabolic product of methacetin or a derivative thereof, in a subject's breath after administering to the subject isotope labeled methacetin or a derivative thereof and wherein the detection and/or evaluation of the liver inflammation is further based on the subject's metabolic product of the methacetin or derivative thereof.

165. The method of claim 156, further comprising measuring a metabolic product of methionine, a salt or a derivative thereof, in a subject's breath after administering to the subject isotope labeled methionine, a salt or a derivative thereof and wherein the detection and/or evaluation of the liver inflammation is further based on the subject's metabolic product of the methionine, the salt or derivative thereof.

166. The method of claim 156, wherein the detection and/or evaluation of the liver inflammation is further based on a physiological and/or medical parameter selected from the group consisting of age, gender, weight, height, waist circumference, blood related parameter, body mass index (BMI) and medication therapy related parameter.

167. The method of claim 156, wherein isotope labeled fatty acid comprises carbon-13, carbon-14, oxygen-18 or any combination thereof.
